(19) United States
(12) Patent Application Publication
(10) Pub. No.: US 2016/0367537 A1
(43) Pub. Date: Dec. 22, 2016

(54) COMPOSITIONS AND METHODS FOR THE TREATMENT AND MANAGEMENT OF STEATOSIS IN HUMAN LIVER

(71) Applicant: WAKE FOREST UNIVERSITY HEALTH SCIENCES, Winston-Salem, NC (US)

(72) Inventor: Lawrence L. Rudel, Winston-Salem, NC (US)

(21) Appl. No.: 14/898,901

(22) PCT Filed: Jun. 18, 2014
(86) PCT No.: PCT/US2014/042997

§ 371 Date: Dec. 16, 2015

Related U.S. Application Data
(60) Provisional application No. 61/836,405, filed on Jun. 18, 2013.

(51) Int. Cl. A61K 31/4433; G01N 33/92; C12N 15/113

(52) U.S. Cl. CPC A61K 31/4433; C12N 15/1137; C12Y 203/01026; G01N 33/92; C12N 2320/30; G01N 2800/52; G01N 2800/7085

(57) ABSTRACT

Compositions and methods for the treatment and management of steatosis are disclosed.

This application claims priority to U.S. Provisional application No. 61/836,405 filed Jun. 18, 2013, the entire contents being incorporated herein by reference as though set forth in full.

Pursuant to 35 U.S.C. §202(c), it is acknowledged that the U.S. Government has rights in the invention described, which was made with funds from the National Institutes of Health, Grant Number, HL-49373.

### FIELD OF THE INVENTION

This invention relates to the fields of hepatic cell health and fatty acid metabolism in humans. More specifically, the invention provides compositions and methods for the treatment and management of fatty liver disorders, particularly those associated with a lysosomal acid lipase (LAL) deficiency.

### BACKGROUND OF THE INVENTION

Several publications and patent documents are cited throughout the specification in order to describe the state of the art to which this invention pertains. Each of these references are incorporated herein as though set forth in full.

Non-alcoholic fatty liver disease (NAFLD) has emerged as an epidemic in the United States and other industrialized countries.(1) Abnormal fat deposition in the liver worsens patient response to viral hepatitis therapy and steatosis has a significant rate of transition to cirrhosis and hepatocellular carcinoma.(2-4) It is estimated that one third of Americans have fatty liver, and that NAFL will eventually supplant hepatitis C as the leading indication for liver transplant in western countries.(5) Additionally, fatty liver disease has additional human health implications, since steatosis has been linked to insulin resistance, obesity, hyperlipidemia, and atherosclerotic heart disease in humans.(6-8) NAFLD is recognized as the main cause of hepatic steatosis, one of the more common liver disorders in the general population.

Studies have identified a number of genetic determinants of hepatic steatosis, which include mutations in the lysosomal acid lipase (NIPA) gene. Such mutations are recognized as contributing factors to the pathogenesis of cholesterol ester storage disease and Wolman disease. Both of these disease states are characterized by a lack of the LAL enzyme which results in massive accumulation of cholesterol ester and triglycerides in the liver, gut and other organs.

Wolman disease, which is the early-onset form of LAL deficiency, is fatal in most instances, typically before one year of age.

Late onset LAL deficiency, commonly referred to as cholesterol ester storage disease (CESD), may lead to cirrhosis of the liver, liver failure and death among children, adolescents and adults. Those diagnosed with CESD appear to be at an increased risk of stroke, due to atherosclerosis, i.e., the accumulation of lipids in the walls of major arteries.

Individuals who suffer from Wolman disease and CESD frequently are undiagnosed because their symptoms are mistaken for those of more prevalent conditions, e.g., NAFLD, non-alcoholic steatohepatitis (NASH) and alcoholic liver disease.

Currently, medical treatment for LAL deficiency disorders involve attempts at managing symptoms. Combination therapy for reducing cholesterol, together with a diet excluding food rich in cholesterol and triglycerides have been effective at reducing some symptoms of CESD. Intravenous nutritional support is sometimes used for Wolman disease patients, if bone marrow transplantation is being considered. See also, Scriver et al., The Metabolic and Molecular Bases of Inherited Disease, Vol. 2, Chap. 82 “Acid Lipase Deficiency: Wolman Disease and Cholesterol Ester Storage Disease”, 2563-87 (1995).

More recently, it has been proposed to treat LAL deficiency disorders by administering stem cells that provide lysosomal enzymes, or by enzyme replacement therapy using a human recombinant lysosomal acid lipase. See. e.g., U.S. Pat. No. 7,927,587 to Blazer et al. and U.S. Pat. No.8,663,631 to Quinn. The therapeutic efficacy of such treatments, however, has yet to be established.

### SUMMARY OF THE INVENTION

In accordance with the present invention, a method for the treatment and or management of hepatic steatosis in a patient in need thereof is disclosed. An exemplary method entails administration of an effective amount of a steroyl-O-acyltransferase inhibitor, the inhibitor being effective to reduce cholesterol ester accumulation, thereby inhibiting or reducing symptoms associated with steatosis. In one embodiment, the inhibitor is an inhibitory nucleic acid molecule. In yet another embodiment, the inhibitor is pyripyoprene A derivative or a pharmaceutically acceptable salts, solvates or hydrates thereof. In yet another embodiment, the SOAT2 inhibitor is delivered in an amount effective to inhibit cholesterol ester accumulation in the liver of such patients.

### BRIEF DESCRIPTION OF THE DRAWINGS

FIG. 1 is a panel of bar graphs showing that the major lipid class present in liver of all clinical groups was triglyceride, and it appeared to systematically increase among the study groups with the highest average being found in the obese, NASH group.

FIG. 2 is series of graphs showing the systematic increases among study groups in the two neutral lipid classes suggesting that mechanisms leading to accumulation of these lipids may be associated.

FIG. 3 is a series of bar graphs showing the results of analysis of the acyl compositions of hepatic triglycerides and cholesterol esters as compared to the same lipid classes in plasma.

FIG. 4 is a series of bar graphs showing that the percentages of CEFA in the liver compared to those percentages in plasma across all study groups by paired t-test were higher in saturated and monounsaturated fatty acids which presumably represents the contribution made by the tissue SOAT2 enzyme.

FIGS. 5A-5D are a series of tables showing unesterified and esterified cholesterol concentrations and contents in the liver, small intestine and spleen in LAL^{-/-} mice given the SOAT2 inhibitor, PRD-125 from 21 to 52 or 53 days of age.

### DETAILED DESCRIPTION OF THE INVENTION

In hepatocytes, cholesterol destined for hepatic storage or secretion in very low density lipoprotein (VLDL) can be esterified by the enzyme steroyl O-acyltransferase 2 (SOAT2), also called acyl-CoA:cholesterol acyltransferase 2 (ACAT2). Mice with SOAT2 gene deletions have low hepatic cholesterol ester (CE) concentrations and are protected from triglyceride accumulation (steatosis) when cholesterol is fed. The decreased concentration of hepatic triglycerides (TG) is coupled to an increased secretion of TG in VLDL. Thus, in mice, SOAT2-derived cholesterol esters may play a central role in promoting storage and limiting secretion of TG by the liver. Accordingly, we hypothesized that in humans, SOAT2-derived cholesterol esters may serve a similar role.

Liver biopsies were obtained at elective laparotomy from 54 patients (37F, 17M) with varying degrees of steatosis (Group I, non-obese, no NAFLD; Group II, non-obese, NAFLD, Group III, obese, No NAFLD; Group IV, Obese, NAFLD; Group V, Obese, NASH). Individuals with non-NAFLD chronic liver disease were excluded. Liver/plasma lipids and plasma glucose homeostasis and hepatic gene expression were measured on intraoperative samples.

Mean hepatic CE accumulation (mg/g Pro) primarily a result of SOAT2 activity, differed significantly (p>0.001) by grouping: Group I, 2.9 ±0.7; Group II, 7.2 ±1.4; Group III, 8.1 ±0.5; Group IV, 11.3 ±1.2; and Group V, 19.6 ±3.2. CE concentration was strongly correlated with accumulation of intrahepatic TG (mg/gPro) (r=0.83, p<0.001) which also differed (p<0.001) by grouping, Group 1, 73 ±25; Group II, 264 ±136; Group III, 143 ±26; Group IV, 419 ±71; Group V, 916 ±232. Age, plasma VLDL, LDL and HDL cholesterol and triglyceride concentrations, plasma glucose, insulin, or fructosamine concentrations, HOMA-IR scores, ALT, AST, and AP, serum albumin, total protein or bilirubin concentrations all were not different by group. Measures of gene expression in hepatic lipid metabolism did not reveal likely mechanisms for the strong associations between neutral lipid classes, but the central feature of NAFLD, triglyceride accumulation and macrosteatosis, was highly related to cholesterol ester availability in spite of the complete lack of hypercholesterolemia among NAFLD groups.

Across a wide range of steatosis in humans, hepatic CE concentrations were strongly correlated with hepatic TG concentrations in NAFLD. By analogy with our findings in experimental animals, the data suggest the possibility of a role for cholesterol ester accumulation in liver as a component in the development of steatosis.

In view of these findings, we propose that steroyl O-acyltransferase 2 (SOAT2) provides a novel therapeutic target for the treatment of liver steatosis since evidence in animals shows that deletion of hepatic SOAT2 is associated with lowered accumulation of hepatic triglyceride (10).

In the intestine, SOAT2 is known to catalyze synthesis of cholesterol esters for transport in chylomicrons thereby facilitating cholesterol absorption (11). The fate of newly absorbed chylomicron cholesterol ester is to be efficiently delivered to the liver where the influx of newly absorbed dietary cholesterol provides substrate for many pathways of cholesterol homeostasis.(12) These include secretion into bile, incorporation into the plasma membrane, and esterification by hepatic SOAT2 resulting in cholesterol esters (CE) that get secreted in very low density lipoproteins (VLDL) or stored in cytoplasmic lipid droplets within hepatocytes. During cholesterol feeding in mice, the amount of hepatic CE storage rises significantly.(10) Conversely, when the murine SOAT2 gene has been deleted or antagonized by a phosphorothioate modified SOAT2-specific antisense oligonucleotide, hepatic triglyceride secretion in VLDL is increased while the triglyceride concentration in the liver is markedly reduced (10). Furthermore, in the absence of SOAT2, the levels of cholesterol and cholesterol ester stored and secreted by the liver remain low, even when dietary cholesterol is fed, so that hepatic steatosis is lessened (10). Thus in mice, SOAT2 in the liver has been found to exert a role in both cholesterol and triglyceride accumulation, a role much more pervasive than just governing esterification of excess cholesterol. In this context, it is important to note that in a study of 9221 participants with 13.3 years of follow-up, 118 diagnoses of cirrhosis and 5 diagnoses of liver cancer were positively linked to higher intakes of dietary cholesterol (13). Clearly, increased dietary cholesterol absorption can lead to a subsequent increase in hepatic cholesterol ester derived from SOAT2, and its association with hepatic triglyceride accumulation, as shown in mice. Accordingly, inhibition of this enzyme provides a new paradigm for the treatment of fatty liver disease in humans.

The results of animal testing conducted by the present inventors, (Unpublished data), indicate that the method of this invention can be used in the treatment of lysosomal acid lipase deficiency, two known forms of which are CESD and Wolman disease. This disease is also commonly referred to as acid lipase disease or acid cholesteryl ester hydrolase deficiency, Type 2. The method of this invention may also be beneficially applied for the treatment of NAFLD, NASH, alcoholic liver disease, cryptogenic cirrhosis, Niemann-Pick disease Type C and Chanarin Dorfman syndrome, Abetalipoproteinemia, Familial hypobetalipoproteinemia, Citrullinemia type II, Familial partial lipodystrophy type 2, Familial partial lipodystrophy type 3, Congenital generalized lipodystrophy, Neutral lipid storage disorder, Cholesterol ester storage disease, Medium-chain acylcoenzyme-A dehydrogenase deficiency, Very long-chain acyl-CoA dehydrogenase deficiency, and Long-chain 3-hydroxyacyl-CoA dehydrogenase deficiency.

### Definitions:

The acronyms “SOAT2 and ACAT2” are used interchangeably to identify an enzyme capable of catalyzing the esterification of cholesterol, as well as oxysterols, with fatty acyl CoA substrates. In addition to being capable of catalyzing the esterification of cholesterol with a fatty acyl CoA substrates, the enzyme encoded by the ACAT-2 gene is incapable of esterifying, at least to any substantial extent, the following substrates: ethanol, retinol, tocopherol, β-sitoserol, lanosterol, vitamins D1 and D2, or diacylglycerol. With respect to fatty acyl CoA substrates, the ACAT-2 polypeptides exhibit the following preference: palmitoyl>linoleoyl>oleoyl>arachindonyl. With ACAT-2 polypeptides, linoleoyl and palmitoyl compete with oleoyl for incorporation into cholesterol esters, but arachindonyl competes less well.

The term “SOAT2/ACAT2 inhibitor” refers to an agent capable of inhibiting the activity of steroyl O-acyltransferase and thereby lowering the concentration of cholesterol ester in the lipid and decreasing symptoms associated with fatty liver disease. A SOAT2 inhibitor may be a competitive, noncompetitive, or irreversible SOAT2 inhibitor. SOAT2 inhibitors of the instant invention include, without limitation, antisense oliogonucleotides which inhibit expression of SOAT2 in humans, siRNA molecules directed to SOAT2 and the compounds disclosed in PCT/JP2008/073501, PCT/JP2011/057336, U.S. patent application Ser. No. 12/810,545 (U.S. Patent Application Publication No. 2011/0184173 and U.S. patent application Ser. No. 13/638,332 (U.S. Patent Application Publication No. 2013/0085163).

One or more supplementary active agents may also be administered with the SOAT2 inhibitor in the practice of this invention. For example, the SOAT2 inhibitor may be administered together, i.e., simultaneously or sequentially, with one or more cholesterol-lowering drugs, known as statins and/or ezetimide or other cholesterol absorption inhibitor.

The term “treatment”, as used herein, refers to therapy that provides relief from or alleviation of the symptoms of lysosomal acid lipase deficiency disorders. This includes managing a patient's symptoms and halting progression of the disorder. Treatment includes slowing, stopping or reversing the development of a lysosomal acid lipase deficiency disorder, and/or the onset of symptoms associated therewith in a patient with, or at risk of developing, lysosomal acid lipase deficiency or a related disorder, i.e., a “patient in need”. For the treatment of lysosomal acid lipase deficiency, the therapy typically includes slowing, stopping or reversing the accumulation of cholesterol ester in liver. Therapy also includes decreasing the amount of accumulated substrate in a patient with lysosomal acid lipase deficiency. Therapy may also include slowing, stopping or reversing symptoms associated with lysosomal acid lipase deficiency.

The expression “therapeutically effective amount” as used herein signifies a non-toxic amount of a SOAT2 inhibitor which is sufficient to limit cholesterol ester acumulation to the extent necessary to provide the desired therapeutic effect in a patient receiving treatment. A therapeutically effective amount minimizes or reverses disease progression associated with the lysosomal acid lipase deficiency. Disease progression may be monitored by clinical observation, laboratory and imaging investigations familiar to a person skilled in the art.

The SOAT2 inhibitor, together with any supplementary active agent(s), may be administered using any amount or any route of administration effective for reducing the symptoms of fatty liver disease. The inhibitor used in practicing the method described herein may be administered topically, enterally or parenterally, such as by subcutaneous injection, intramuscular injection, interperitoneal injection, intravenous infusion, transdermal patch or the like. The PRD125 compound is effective orally and does not need booster injections, etc.

The SOAT2 inhibitor is preferably formulated in dosage unit form, together with supplementary active agent(s) and auxiliary agent(s), if any, in dose unit form for ease of administration and uniformity of dosage. “Dosage unit” as used herein refers to a physically discrete unit of one or more SOAT2 inhibitors suitable for the patient to be treated. Each dosage should contain the quantity of active agent calculated to produce the desired therapeutic effect when administered as such, or in association with the selected pharmaceutical carrier medium. The appropriate quantity of therapeutic agent to be included in a specific dosage unit can be determined by procedures well known in the art, and is such as to achieve an appreciable change in the disease state. Those skilled in the art can readily measure the levels of a small molecule inhibitor of cholesterol oleate in a plasma or target tissue. The concentration of the inhibitor in these samples can be compared with a predetermined inhibitory amount established in vitro to establish therapeutic efficacy.

The appropriate dosage unit will be administered from once per day up to about 4 times per day. While a dosage schedule of once-a-day is preferred, a more frequent dosing schedule may be required so as to maintain a therapeutically effective serum concentration of the SOAT2 inhibitor.

The effective amount of a given therapeutic agent and its optimal mode of administration are determined in accordance with established medical standards, taking into account the species, age, gender, weight and health of the patient, the nature and severity of the clinical condition being treated, the particular therapeutic agent being administered, its route of administration, the judgment of the attending medical professional, and the like. The term “patient” as used herein refers to animals including mammals, preferably humans.

While not wishing to be confined to any particular theory as to the mechanism of action of the SOAT2 inhibitor used in practicing the present invention, it is believed that the observed inhibitory effect on hepatic lipid accumulation is due to inhibition of hepatic cholesterol esterification.

The following materials and methods are provided to facilitate the practice of the present invention.

### Methods

After informed consent, 55 patients undergoing elective abdominal surgery (e.g. cholecystectomy, weight loss surgery, gastric fundoplication, and hernia repair) at Wake Forest University Baptist Hospital were enrolled for the intraoperative collection of a wedge liver biopsy, core needle biopsy, and 10 ml of whole blood. Patients were excluded for a history of chronic liver disease other than NAFLD, a history of malignancy other than non-melanomatous skin cancer, an INR greater than 1.8, the need for therapeutic anticoagulation after surgery, a history of chronic inflammatory diseases including but not limited to rheumatoid arthritis, psoriasis, lupus, sarcoidosis, and inflammatory bowel disease, consumption of greater than or equal to 105 grams of ethanol per week , greater than or equal to 45 grams of ethanol in a given day, and refusal to participate. One normal weight patient was excluded from further study when hyperlipidemia was identified.

Wedge biopsies of liver ranging from 250-500 mg were rinsed with normal saline and immediately snap frozen in liquid nitrogen in the operating room before subsequent storage at −80° F. Liver lipid concentrations were measured after performing chloroform-methanol extraction on approximately 100 mg of liver tissue according to the Bligh and Dyer method (15). An aliquot of liver lipid extract was then solubilized for total and free cholesterol quantification by enzymatic assays as previously described (16, 17). The amount of cholesterol ester was calculated by subtracting free cholesterol from the total cholesterol, and multiplying the difference by 1.67 for conversion to CE mass.

Intraoperative core needle liver biopsies were obtained with an 18 gauge spring loaded MC1820 Bard™ biopsy device. Punch biopsies were immediately placed in 10% buffered formalin and submitted for steatosis, steatohepatitis, and fibrosis evaluation by a single expert pathologist using a standardized scoring system for reference (18). A semi quantitative estimate of macrosteatosis was made by point counting hepatocytes at 400× optical magnification. The hepatocytes of five, non-overlapping fields that did not include a portal tract were hand counted, yielding a percentage of macrosteatotic, microsteatotic, and normal hepatocytes. An overlying grid of 35 μm² boxes was used to assist counting. Lipid droplets that displaced the hepatocyte nucleus and/or measured ≧15 μm in diameter were identified as macrosteatotic. Degrees of macrosteatosis were graded according to convention: None: 0%, Grade 1: 1-33%, Grade 3: 33-66% (18). Zonal hepatocyte ballooning/disarray was graded on a scale of 0 to 2 and lobular necroinflammation was graded on a scale of 0 to 3. Subjects with a macrosteatosis grade of ≧1 were classified into the NAFLD phenotype. The individual grades of macrosteatosis, ballooning, and lobular necroinflammation were combined to calculate a NAFLD activity score (NAS) on each subject. A subject with a NAS 5 was categorized as the NASH phenotype (19). Subjects were then placed into one of five clinical groups based on BMI and NAFLD phenotype: (Group I, non-obese, no NAFLD; Group II, non-obese, NAFLD; Group III, obese, No NAFLD; Group IV, Obese, NAFLD; Group V, Obese, NASH). Histopathological assessments for all liver specimens were made by a single expert hepatopathologist.

Blood samples were collected in EDTA (purple top) tubes and stored on ice before separation of plasma from red cells by centrifugation. Plasma was then stored at −80° F. Plasma lipid concentrations [TG, total plasma cholesterol (TPC), CE] and quantification of discrete fatty acid (FA) species were determined with enzymatic assay and/or gas chromatography as previously described in detail (16, 17, 20).

As indicators of glucose tolerance, fasting blood glucose (FBG) (mg/dL) and serum fructosamine, were measured with commercially available reagents (Roche). Fasting insulin (FI) level (mmol/dL) was determined with an ELISA method (Mercodia™), allowing for the calculation of a homeostasis model assessment (HOMA) of insulin resistance [(FBG×FI)/405] (21, 22).

SOAT2 activity was assayed in each of the bariatric liver samples. Briefly, hepatic microsomal membranes were isolated from total liver homogenates made from snap frozen liver samples as previously described (23, 24). Assays for SOAT2 activity were performed in duplicate using 50 μg aliquots of total protein in liver microsomal membrane suspensions (23).

Total RNA from approximately 30-50 mg of human liver was extracted using 2 ml Trizol (Life Technologies) according to the manufacturer's protocol. The resulting RNA pellet was suspended in 300 μl DEPC-treated water. An aliquot was removed to determine concentration by spectroscopy at 260 nm and 1 μg was run on a non-denaturing gel to verify RNA integrity. One microgram of total RNA was reverse transcribed into cDNA using qScript (Quantas Bioscience) according to the manufacturers recommendations. The cDNA was then diluted 1:10 with DEPC-treated water.

For realtime PCR, 5 μl of each cDNA was run in duplicate using FastStart Universal Sybr Green (Rox) (Roche) with a final concentration of primers at 250 nM. The reaction was run at 95° C. 10″, 95° C. 10″, 60° C. 30″ for 40 cycles on a 7500 Fast Realtime per detection system (Applied Biosciences). Ct values from PCR were acquired with background set to cycles 5-12 and a threshold of 0.2. All Ct values for specific genes were normalized to the Ct for Gapdh.

Epidemiological data on patient age, sex, comorbidities (diabetes mellitus and hyperlipidemia), medication use (anti-hyperlipidemics, insulin, insulin secretagogues, and insulin sensitizers) were collected from the medical record and patient questionnaires.

### Statistical Analysis

Comparisons among groups have been made using One-Way Analysis of Variance for continuous variables and Tukey-Kramer HSD posthoc test. Correlations have been evaluated using the Pearson product-moment correlations for each pair of X and Y variables. Differences were considered to be significant with p value <0.05. All analyses were performed using JMP statistical software by SAS Institute (version 5.0.1.2, SAS Institute, Cary, N.C.)

Sample size estimations were performed by testing the hypothesis that the mean levels for the control group vs affected groups would be between 1.25 and 1.5 for individuals with NAFLD and NASH, respectively. Assuming a standard deviation of plus or minus 25% for any group variable and attempting to reach a power of detection of 80% to detect significance at the 5% confidence level, at least 6 subjects would be required for any group. While we could not be sure of how many patients could be recruited for any one group, we estimated that an overall group size of at least 55 should permit at least n=6 in any one group thereby permitting us to make appropriate estimations of significance where they occur.

The following examples are provided to illustrate certain embodiments of the invention. They are not intended to limit the invention in any way.

### EXAMPLE I

### The Human SOAT2 Enzyme Provides a Candidate Target for NAFLD Therapies

From the 54 patients evaluated, the degree of liver disease was classified with a NAFLD activity score (NAS). Accordingly, 7 were obese with NASH, 24 were obese with NAFLD, and 13 were obese without NAFLD. A 7 patient, non-obese control population also was studied as were 3 patients with NAFLD that were nonobese. The demographic, clinical and laboratory data for these five study groups are shown in Table 1. Except for body weight and BMI, the 5 study groups had comparable values for categories listed including age, tissue enzymes, liver function tests, and measures of glycemia. It should be noted that plasma lipoprotein cholesterol concentrations including VLDL, LDL and HDL cholesterol were also comparable among groups. Previous studies have characterized patient populations where this was not the case (13) and it is important to realize that the hepatic phenotype in fatty liver disease as found here is not necessarily associated with hyperlipidemia.

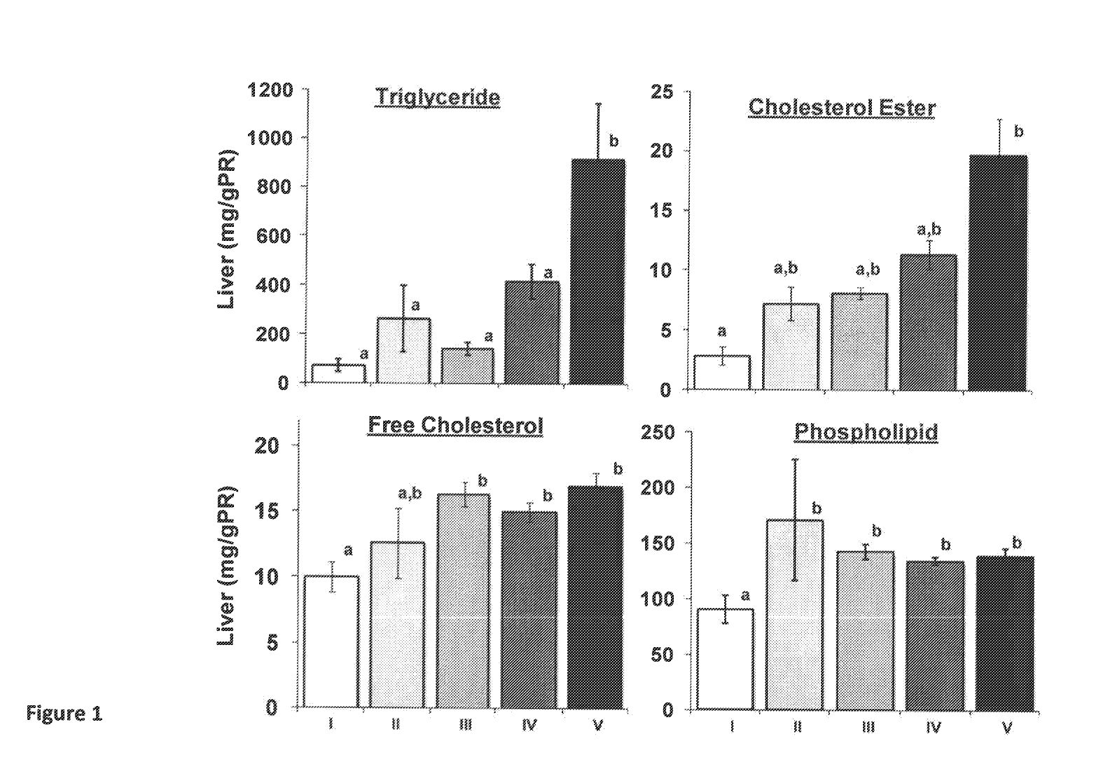

Hepatic lipid concentrations and histologic evaluations were obtained for each of the liver biopsies. The data in FIG. 1 show that the major lipid class present in liver of all clinical groups was triglyceride, and it appeared to systematically increase among the study groups with the highest average being found in the obese, NASH group. The other neutral lipid class is cholesterol ester. and it showed a similar trend to triglyceride and was also highest on the mean in the obese, NASH group. The data in Table 2 show the comparisons among patient groups in hepatic macrosteatosis, perhaps the more common measure of steatosis, and the two neutral lipids measured chemically. Again, the highest values were found in the obese NASH patients. The concentrations of the membrane predominant lipids, free cholesterol and phospholipid, were not different among the clinical groups studied here (FIG. 1) and were not highly correlated to the degree of macrosteatosis or triglyceride concentration (data not shown).

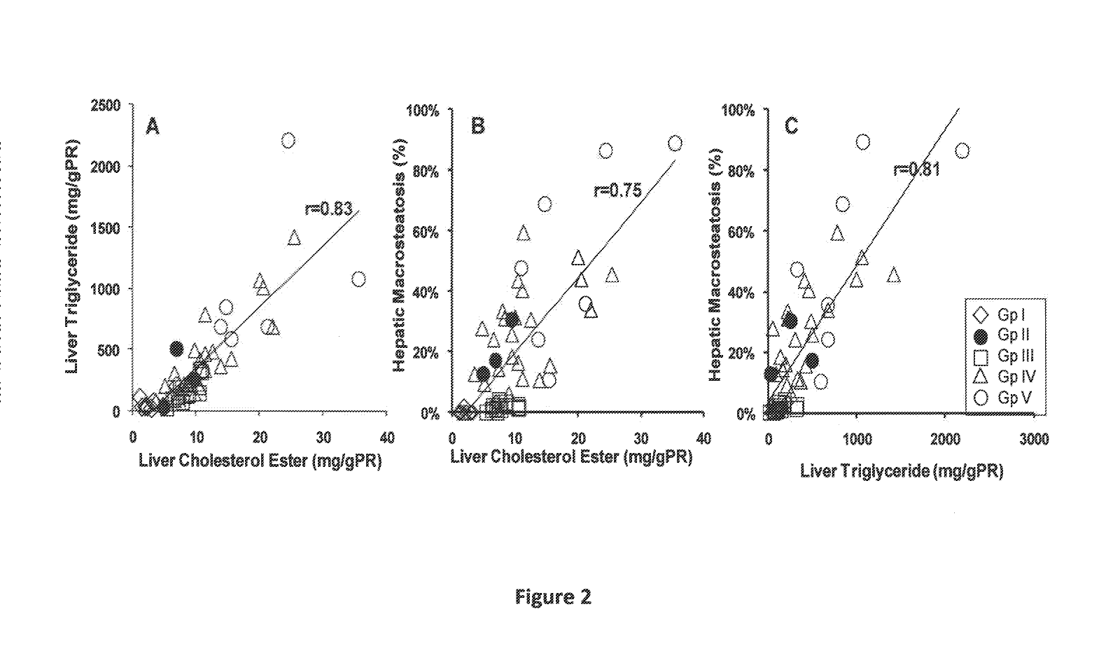

The systematic increases among study groups in the two neutral lipid classes (FIG. 2) suggest that mechanisms leading to accumulation of these lipids may be associated. As per our hypothesis based on murine data (10) the correlation (r) of intrahepatic cholesterol ester concentration (mg/g protein) to both intrahepatic triglyceride concentration (mg/g protein) and degree of histopathological macrosteatosis (%) were highly significant (r=0.83, p<0.001 and r=0.75, p<0.001, respectively) (FIG. 2A, B) as was the correlation between hepatic triglyceride concentration and macrosteatosis (FIG.1C) r=0.81, p<0.001). These data demonstrate for the first time in human patients, a high degree of association between hepatic steatosis, triglyceride and CE i.e. SOAT2 enzyme product accumulation.

TABLE 1
Patient Clinical Data by Liver Phenotype

| | Group I | Group II | Group III | Group IV | Group V | |
|---|---|---|---|---|---|---|
| | Non-Obese | Non-Obese | Obese | Obese | Obese | |
| Parameter | No NAFLD | NAFLD | No NAFLD | NAFLD | NASH | Probability |
| n | 7 | 3 | 13 | 24 | 7 | |
| Age (yrs) | 51.9 ± 5.5 | 45.7 ± 5.8 | 46.2 ± 2.6 | 49.8 ± 2.5 | 41.7 ± 4.0 | NS |
| Wt (lbs) | 161 ± 11 | 178 ± 19 | 254 ± 17 | 255 ± 9 | 313 ± 39 | |
| BMI (kg/m²) | 26.4 ± 1.0 | 27.8 ± 1.0 | 39.6 ± 1.9 | 43.0 ± 1.2 | 53.1 ± 6.2 | |
| Male/female | 3/4 | 2/1 | 5/8 | 6/18 | 1/6 | |
| Plasma TG (mg/dl) | 86 ± 13 | 107 ± 27 | 112 ± 13 | 134 ± 12 | 127 ± 24 | NS |
| Plasma TPC | 165 ± 12 | 167 ± 16 | 187 ± 15 | 165 ± 7 | 150 ± 18 | NS |
| VLDLC (mg/dl) | 5.0 ± 0.9 | 8.6 ± 3.4 | 10.9 ± 2.1 | 11.4 ± 1.5 | 9.3 ± 2.2 | NS |
| LDLC (mg/dl) | 104 ± 8 | 117 ± 8 | 130 ± 13 | 111 ± 8 | 104 ± 18 | NS |
| HDLC (mg/dl) | 57 ± 6 | 41 ± 5 | 46 ± 5 | 42 ± 3 | 36 ± 3 | NS |
| AST (U/L) | 27.4 ± 5.5 | 17.0 ± 1.0 | 29.6 ± 2.7 | 26.3 ± 1.7 | 45.5 ± 11.5 | NS |
| ALT (U/L) | 28.8 ± 5.0 | 18.5 ± 6.5 | 37.7 ± 6.0 | 31.5 ± 3.4 | 45.3 ± 16.4 | NS |
| AP (U/L) | 53.2 ± 6.0 | 68.5 ± 0.5 | 76.1 ± 6.0 | 72.0 ± 3.6 | 69.2 ± 6.0 | NS |
| F Glu (mg/dl) | 92 ± 13 | 109 ± 17 | 108 ± 8 | 123 ± 8 | 129 ± 19 | NS |
| Insulin (uLu/ml) | 6.5 ± 4.0 | 15.8 ± 8.7 | 9.1 ± 2.0 | 8.5 ± 2.0 | 12.1 ± 5.0 | NS |
| HOMA | 1.9 ± 1.4 | 4.8 ± 2.7 | 2.3 ± 0.5 | 3.1 ± 1.1 | 5.1 ± 2.6 | NS |
| Fructosamine | 194 ± 14 | 186 ± 0 | 185 ± 9 | 188 ± 11 | 190.6 ± 16.0 | NS |
| Platelet Ct (×1000) | 237 ± 22 | 229 ± 27 | 292 ± 18 | 258 ± 13 | 287 ± 47 | NS |
| Bilirubin (mg/dl) | 0.7 ± 0.1 | 0.8 ± 0.1 | 0.7 ± 0.1 | 0.8 ± 0.1 | 0.6 ± 0.1 | NS |

TABLE 2

| | Group I | Group II | Group III | Group IV | Group IV | |
|---|---|---|---|---|---|---|
| | Non-Obese | Non-Obese | Obese | Obese | Obese | |
| Parameter | No NAFLD | NAFLD | No NAFLD | NAFLD | NASH | Probability |
| N | 7 | 3 | 13 | 24 | 7 | |
| Liver Triglyceride (mg/g PR) | 73 ± 25^{a} | 264 ± 136^{a} | 143 ± 26^{a} | 419 ± 71^{a} | 916 ± 232^{b} | <0.0001 |
| Liver Cholesterol Ester (mg/g PR) | 2.9 ± 0.7^{a} | 7.2 ± 1.4^{a,b} | 8.1 ± 0.5^{a,b} | 11.3 ± 1.2^{b} | 19.6 ± 3.2^{b} | <0.0001 |
| Liver Macrosteatosis (%) | 1 ± 1^{a} | 20 ± 5^{a,b} | 2 ± 0.4^{a,b} | 26 ± 3^{b} | 52 ± 12^{c} | <0.0001 |

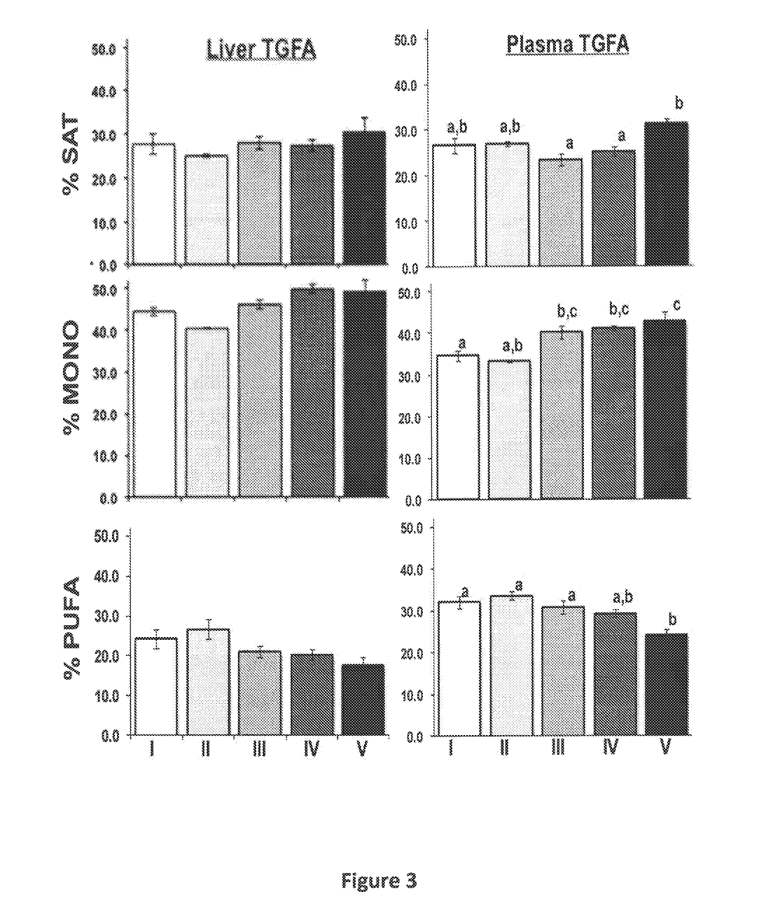

One possible influence on hepatic lipid concentrations may be the availability of individual fatty acid species; therefore we analyzed the acyl compositions of hepatic triglycerides and cholesterol esters as compared to the same lipid classes in plasma. There was no major trend for a difference by NAFLD phenotype in acyl compositions of hepatic triglycerides with about 30% of the fatty acyl species being saturated, 20% being polyunsaturated and 50% being monounsaturated (FIG. 3).

The pattern among study groups for plasma TGFA was somewhat different to that for liver (FIG. 3) although in plasma triglycerides, the obese NASH group had significantly higher percentages for saturated and monounsaturated acyl species (and lower percentages of PUFA species) than for the nonobese control group. Overall, by paired t test, the percentage of PUFA in plasma TG was higher than the percentage in hepatic TG and the percentage of MUFA was lower in plasma TG.

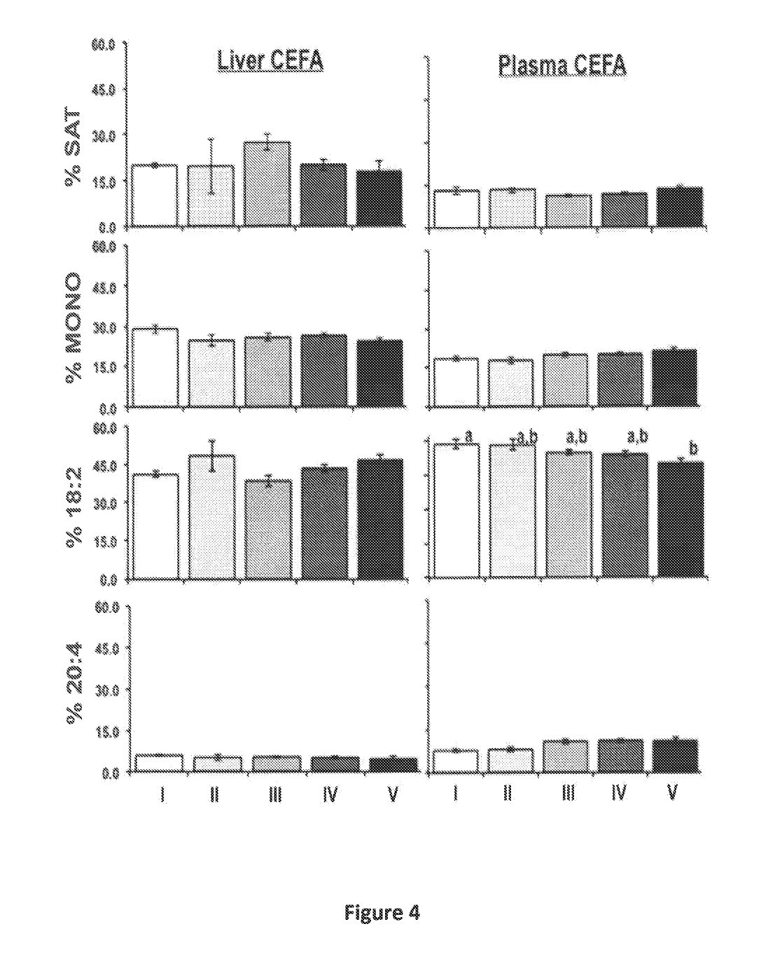

The data for hepatic and plasma cholesterol ester acyl compositions are shown in FIG. 4. By paired t test, the percentages among fatty acids in liver CE are different than those in plasma CE. A much higher percentage of fatty acyl species were polyunsaturated in plasma cholesterol esters than in triglycerides, presumably reflecting the acyl specificity of the plasma lecithin:cholesterol acyltransferase enzyme that typically is responsible for synthesis of the majority of plasma cholesterol esters. The percentage composition of hepatic CE was not different among study groups. The percentage of fatty acids in plasma CE that were polyunsaturated was significantly lower in the NASH group than in the control group, and while this could represent an increased contribution of SOAT2 derived CE in this group, the difference was quite small. The percentages of CEFA in the liver compared to those percentages in plasma across all study groups by paired t-test were higher in saturated and monounsaturated fatty acids which presumably represents the contribution made by the tissue SOAT2 enzyme, (FIG. 4). Overall, the acyl composition data indicates that the accumulation of TG and CE in NAFLD is not likely a result of a major difference in availability of any one acyl class.

The observation of a systematic accumulation of CE in the liver led us to measure the levels of various sterol intermediates to assess whether or not the hepatic accumulation was more related to cholesterol synthesis vs intestinal cholesterol absorption. The data in Tables 3A and 3B either shown as sterol concentration in plasma or sterol concentration corrected for plasma cholesterol concentration, show that no differences in the concentrations of cholesterol synthesis intermediates lathosterol and desmosterol were present suggesting that cholesterol synthesis rates were likely comparable among all NAFLD study groups. On the other hand, there were lower amounts of the plant sterols campesterol and sitosterol in the plasma of the NASH and NAFLD patients compared to the control nonobese group, suggesting that cholesterol absorption was lower in the individuals with NAFLD. Thus, the higher hepatic concentrations of CE found in the Obese NAFLD and NASH patients (FIG. 1) is likely not a result of a higher degree of dietary cholesterol absorption. This outcome suggests that hepatic cholesterol ester accumulation occurs independent of increased intestinal cholesterol absorption. Further, it is not likely due to increased hepatic cholesterol availability via increased synthesis, a conclusion consistent with the lack of any increase in the expression level of HMG CoA reductase.

TABLE 3A

| Group | | Lathosterol | Desmosterol | Campesterol | Sitosterol |
|---|---|---|---|---|---|
| | N | ng/ml | | | |
| I. Non-obese, No NAFLD | 7 | 1627 ± 368 | 807 ± 122 | 5586 ± 216^{a} | 4741 ± 208^{a} |
| II. Non-obese, NAFLD | 3 | 2226 ± 1220 | 899 ± 80 | 4436 ± 142^{a, b} | 3651 ± 215^{a, b} |
| III. Obese, No NAFLD | 13 | 2260 ± 337 | 904 ± 103 | 3907 ± 594^{a, b} | 3700 ± 406^{a, b} |
| IV. Obese, NAFLD | 24 | 1947 ± 223 | 810 ± 59 | 2998 ± 283^{b} | 3005 ± 199^{b} |
| V. Obese, NASH | 7 | 2350 ± 390 | 798 ± 139 | 2695 ± 523^{a, b} | 2686 ± 355^{b} |

TABLE 3B

| Group | | Lathosterol | Desmosterol | Campesterol | Sitosterol |
|---|---|---|---|---|---|
| | N | ng/μg cholesterol | | | |
| I. Non-obese, No NAFLD | 7 | 100.4 ± 23.0 | 49.7 ± 7.8 | 349 ± 28^{a} | 292 ± 12^{a} |
| II. Non-obese, NAFLD | 3 | 140.4 ± 81.1 | 54.2 ± 4.3 | 272 ± 31^{a, b} | 223 ± 27^{a, b} |
| III. Obese, No NAFLD | 13 | 115.4 ± 18.9 | 45.2 ± 5.1 | 196 ± 32^{b} | 190 ± 25^{b} |
| IV. Obese, NAFLD | 24 | 118.0 ± 12.4 | 49.3 ± 2.9 | 182 ± 16^{b} | 186 ± 12^{b} |
| V. Obese, NASH | 7 | 151.3 ± 17.8 | 51.0 ± 5.1 | 179 ± 24^{b} | 183 ± 16^{b} |

Despite being the only known enzyme responsible for cholesterol ester synthesis in hepatocytes, SOAT2 mRNA expression was not significantly correlated with intrahepatic CE (r=0.27) or TG (0.3) concentrations in our study groups. Further, similar levels of SOAT2 activity were measured across all groups, indicating that SOAT2, while apparently present in almost all patients, is not a highly regulated enzyme.

### Discussion

The significance of cholesterol intake and metabolism in the development of clinical atherosclerosis is well established; however, recent work with the SOAT2 enzyme in a murine model raises the question of a cholesterol mediated pathway in hepatic steatosis. Here we have shown that cholesterol ester is closely associated with human intrahepatic triglyceride content, histological macrosteatosis, and the NASH clinical phenotype.

Excess hepatic cholesteryl ester as compared to healthy controls has been reported in NAFLD patients by Puri et al; and Innodou et al have recently reported that liver related morbidity in 9,221 patients with 13.3 years of follow up correlated to excess dietary cholesterol intake.(24, 25) In this context, the strong statistical correlation of the steroyl O-acyltransferase 2 (SOAT2) enzyme product, hepatic cholesteryl ester, to human hepatic triglyceride, histological macrosteatosis, and steatohepatitis as seen in this study supports our original hypothesis that cholesterol ester may represent a novel promoter for liver steatosis and perhaps inflammation. In fact, liver cholesterol ester is more closely associated with liver steatosis in this cohort than commonly reported patient factors like BMI, glucose intolerance, or plasma hyperlipidemia. This contrasts with recent work by Min et al that suggests it is free cholesterol not CE that is associated with progressively worse NAFLD phenotypes.(13)

It is not clear how a seemingly unrelated hepatic cholesterol pathway might exert such influence over human hepatic triglyceride storage. Work by Alger et al examining liver triglyceride handling by the liver in an SOAT2 knockout (KO) mouse indicates that increased hepatic export of liver triglyceride—not altered TG synthesis—in ACAT2/cholesteryl ester depleted liver may be responsible for this phenomenon.(10) In a series of mouse liver perfusion experiments, existing hepatic triglyceride was pre-labeled with tritiated (H³) oleic acid and newly synthesized TG was marked with Carbon¹⁴ (C¹⁴). The results demonstrated equal mobilization of de novo synthesized (C¹⁴ labeled) and pre-existing (H³ labeled) hepatic TG in the liver perfusate of ACAT2 wild type (WT) and KO mice. One result of this altered TG mobilization is that serum very low density lipoprotein (VLDL) rich in TG is also increased in mice depleted of hepatic CE. Fujita et al. have reported similar trends in human subjects in which impaired VLDL synthesis and transport are associated with findings of NAFL and NASH.(26) We hypothesize that CE may compete with TG for binding sites hepatocyte hydrolase(s) necessary for TG mobilization. Several of the known hydrolysis enzymes (e.g. hormone sensitive lipase) are recognized to have activity on both TG and CE, although the relative affinities have not been fully described.(27) As in other studies, plasma VLDL was elevated in obese subjects with increased intrahepatic triglyceride.(28) However, this doesn't necessarily indicate efficient export of liver fatty acids. Work by Fabbrini et al demonstrate a linear relationship between intrahepatic liver fat and VLDL secretion in obese patients without NAFLD (12.) Whereas, in their ability to export fatty acids, NAFLD patients appear to plateau independent of percent liver triglyceride content.

Our study used the SOAT2 enzyme product (hepatic cholesteryl ester) as a primary endpoint, but we were interested to note our subjects expressed relatively impressive amounts of the ACAT2 enzyme protein. Although, the individual degree of expression did not correlate significantly liver CE, ACAT2 protein was associated with elevated BMI, as was liver CE. The properties of ACAT2 activity in vivo are ill-defined. It may be that uninvestigated factors, particularly diet and its effects on the intrahepatic acyl CoA substrate “pool,” dictates ACAT2 kinetics.

NAFLD is a highly prevalent, complex disease process influenced by intersecting patient and environmental factors. Currently, exercise and nutrition management are the foundation of NAFLD therapy with bariatric surgery as an emerging option. No drug therapies targeted to reverse fatty liver are currently approved for use by the Food and Drug Administration. Given the SOAT2 knockout mouse data and our supporting human data suggesting that liver CE hinders liver TG export and is associated with NASH, the human SOT2 enzyme provides a candidate target for future NAFLD therapies.

### EXAMPLE 2

### Treatment of Fatty Liver Disease with SOAT2/ACAT2 Inhibitors

Anti-sense oligonucleotide therapies have successfully limited steatosis in murine models. Accordingly, similar approaches can be employed in ACAT2 directed therapy for non-alcoholic fatty liver disease in humans.

In another approach pyripyropene derivatives having ACAT2-inhibiting activity can also be utilized to treat of manage steatosis in human patients. Such derivatives include, without limitation those described in PCT/JP2008/073501, and U.S. patent application Ser. No. 12/810,545 assigned to the Kitasato Institute, each of which is incorporated herein by reference.

Additional inhibitors can be identified using the screening assays set forth in a research article co-authored by the present inventor which appear in The Journal of Lipid Research, 45, 378-386 (2004).

### EXAMPLE 3

### Study of Mouse Model of Lysosomal Acid Lipase Deficiency

In the present example, hepatic lipid concentrations and content in a lysosomal acid lipase deficient (LAL^{-/-}) mouse model were determined in the presence and absence of selective SOAT2 inhibitors. The data are for male lysosomal acid lipase deficient mice on a FVB background. The mice were fed a chow diet for 21 days. During the subsequent 52 or 53 days, 5 mice were fed the chow diet without drug and 3 of 5 to be studied were fed a chow diet supplemented with PRD125 at a dose of 10 mg/kg/day. Animals were ex-sanguinated at 52 days and the blood plasma was collected in EDTA and frozen in liquid nitrogen. The livers, small intestine, and spleen were flushed with saline and rinsed, then also snap frozen in liquid nitrogen. All organs were weighed, and after freezing, approximately 25 mg of tissue was extracted in chloroform methanol, 2:1, for subsequent lipid determinations. Free and total cholesterol was measured enzymatically and esterified cholesterol was determined by difference. Aliquots of the terminal plasma were used for measurement of tissue enzymes.

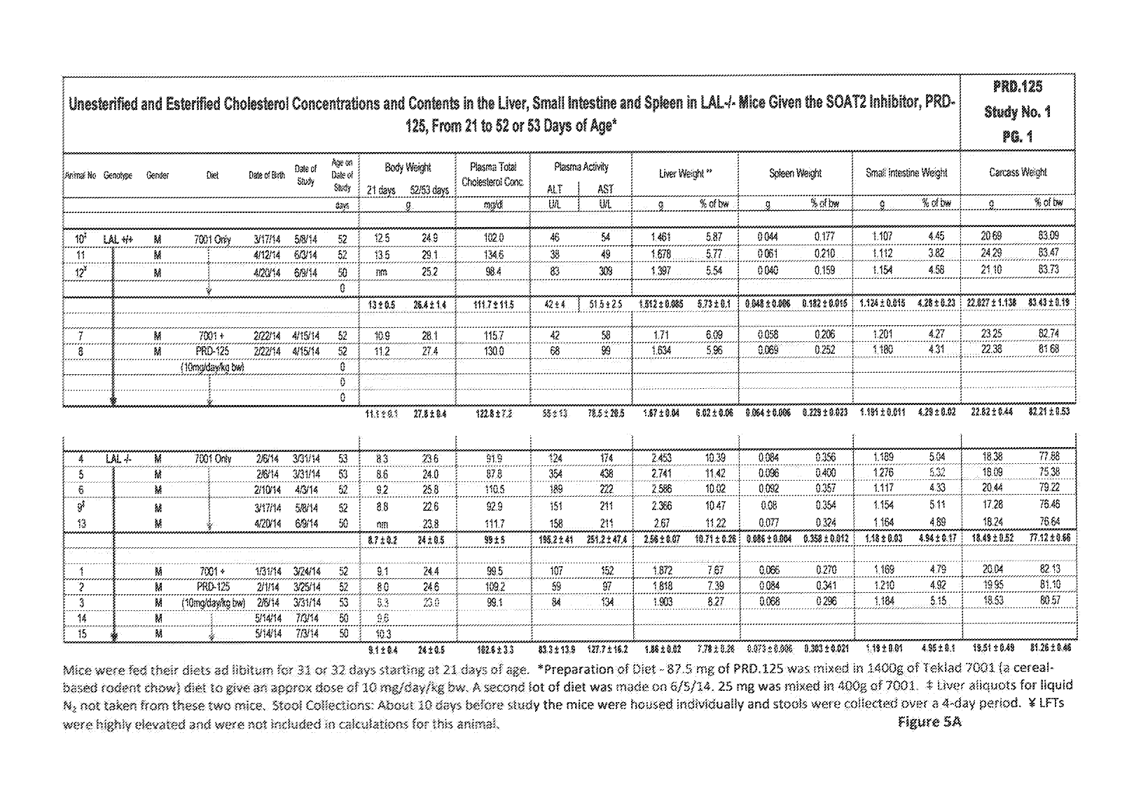

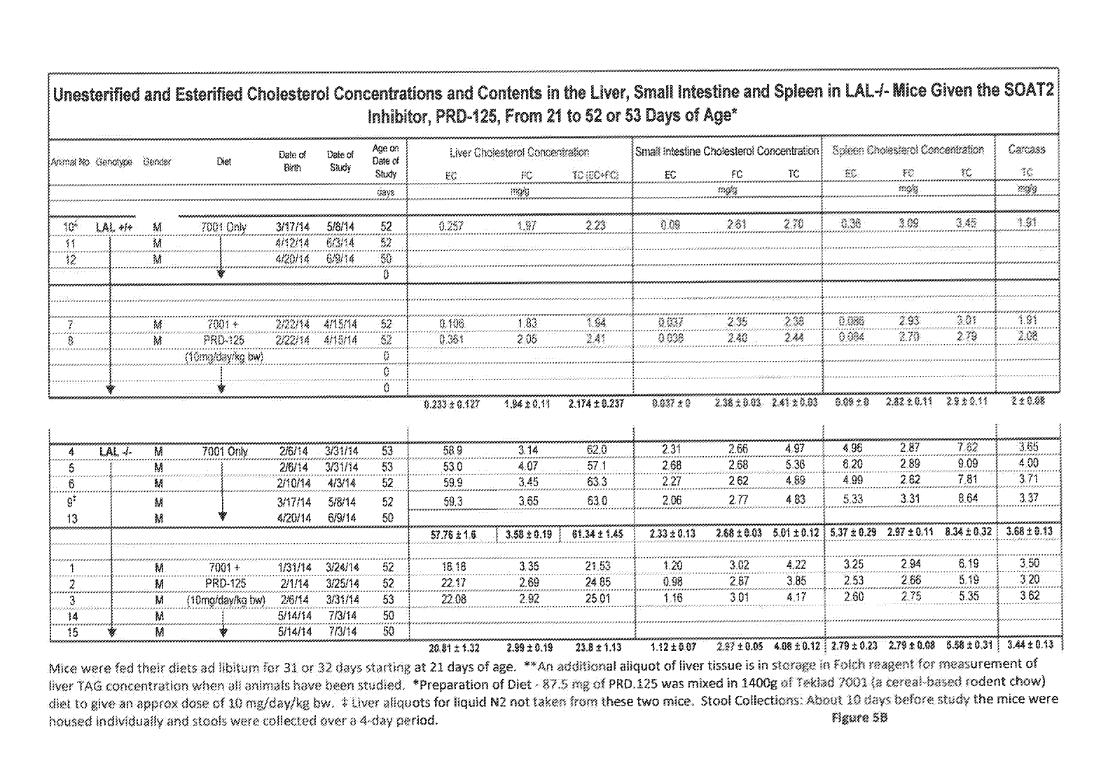

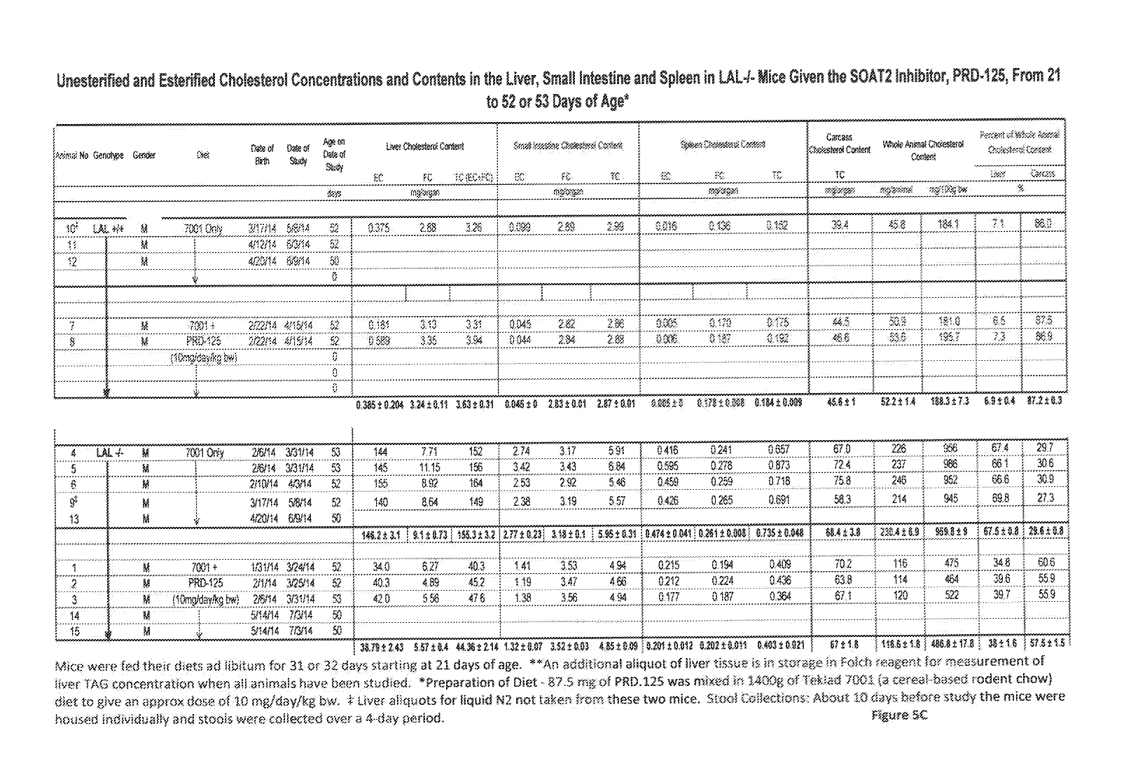

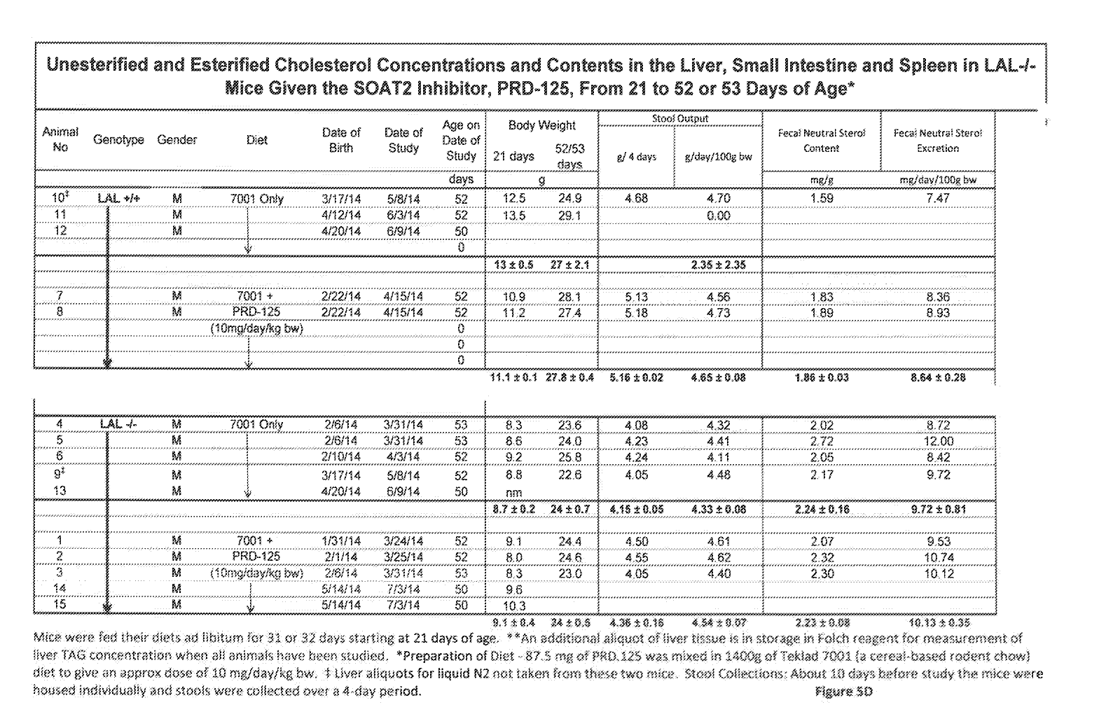

The data show that in the presence of a SOAT2 inhibitor, not only are liver cholesterol esters significantly reduced but serum enzyme levels are also reduced indicating that the response is rather robust. Mice were studied starting at 21 days of age and studied for 52 to 53 days. Treatment with PRD125 was at 10 mg/kg/day and the compound was given as a diet admixture. This inhibitor is over 5000-fold more specific to SOAT2 compared to SOAT1, and it is stable in the diet and is easily absorbed. FIGS. 5A-FD show that cholesterol content is approximately 146 mg/liver in untreated mice and only 39 mg/liver in the treated mice which is a 65% decrease—a very large change demonstrating the promise of this therapeutic approach for the treatment of fatty liver disease.

### REFERENCES

1. Flegal, K. M., Carroll, M. D., Ogden, C. L., and Johnson, C. L. 2002. Prevalence and trends in obesity among US adults, 1999-2000. Jama 288:1723-1727.

2. Adinolfi, L. E., Gambardella, M., Andreana, A., Tripodi, M. F., Utili, R., and Ruggiero, G. 2001. Steatosis accelerates the progression of liver damage of chronic hepatitis C patients and correlates with specific HCV genotype and visceral obesity. Hepatology 33:1358-1364.

3. Nair, S., Mason, A., Eason, J., Loss, G., and Perrillo, R. P. 2002. Is obesity an independent risk factor for hepatocellular carcinoma in cirrhosis? Hepatology 36:150-155.

4. Wanless, I. R., and Lentz, J. S. 1990. Fatty liver hepatitis (steatohepatitis) and obesity: an autopsy study with analysis of risk factors. Hepatology 12:1106-1110.

5. Browning, J. D., Szczepaniak, L. S., Dobbins, R., Nuremberg, P., Horton, J. D., Cohen, J. C., Grundy, S. M., and Hobbs, H. H. 2004. Prevalence of hepatic steatosis in an urban population in the United States: impact of ethnicity. Hepatology 40:1387-1395.

6. Marchesini, G., Bugianesi, E., Forlani, G., Cerrelli, F., Lenzi, M., Manini, R., Natale, S., Vanni, E., Villanova, N., Melchionda, N., et al. 2003. Nonalcoholic fatty liver, steatohepatitis, and the metabolic syndrome. Hepatology 37:917-923.

7. Xirouchakis, E., Sigalas, A., Manousou, P., Calvaruso, V., Pleguezuelo, M., Corbani, A., Maimone, S., Patch, D., and Burroughs, A. K. 2008. Models for non-alcoholic fatty liver disease: a link with vascular risk. Curr Pharm Des 14:378-384.

8. Gastaldelli, A., Kozakova, M., Hojlund, K., Flyvbjerg, A., Favuzzi, A., Mitrakou, A., and Balkau, B. 2009. Fatty liver is associated with insulin resistance, risk of coronary heart disease, and early atherosclerosis in a large European population. Hepatology 49:1537-1544.

9. Bult, M. J., van Dalen, T., and Muller, A. F. 2008. Surgical treatment of obesity. Eur J Endocrinol 158:135-145.

10. Alger, H. M., Brown, J. M., Sawyer, J. K., Kelley, K. L., Shah, R., Wilson, M. D., Willingham, M. C., and Rudel, L. L. 2010. Inhibition of acyl-coenzyme A:cholesterol acyltransferase 2 (ACAT2) prevents dietary cholesterol-associated steatosis by enhancing hepatic triglyceride mobilization. J Biol Chem 285:14267-14274.

11. Nguyen, T. M., Sawyer, J. K., Kelley, K. L., Davis, M. A., and Rudel, L. L. 2012. Cholesterol esterification by ACAT2 is essential for efficient intestinal cholesterol absorption: evidence from thoracic lymph duct cannulation. J. Lipid Res 53: 95-104

12. Goodman, D. S. 1965. Cholesterol ester metabolism. Physiol Rev. 45: 747-839.

13. Ioannou, G. N., Morrow, 0.B., Connole, M. L., and Lee, S. P.2009. Association between dietary nutrient composition and the incidence of cirrhosis or liver cancer in the United States population. Hepatology, 50:175-184.

14. Min, H. K., Kapoor, A., Fuchs, M., Mirshahi, F., Zhou, H., Maher, J., Kellum, J., Warnick, R., Contos, M. J., and Sanyal, A. J. Increased hepatic synthesis and dysregulation of cholesterol metabolism is associated with the severity of nonalcoholic fatty liver disease. Cell Metab 15:665-674.

15. Bligh, E. G., and Dyer, W. J. 1959. A rapid method of total lipid extraction and purification. Can J Biochem Physiol 37:911-917.

16. Temel, R. E., Lee, R. G., Kelley, K. L., Davis, M. A., Shah, R., Sawyer, J. K., Wilson, M. D., and Rudel, L. L. 2005. Intestinal cholesterol absorption is substantially reduced in mice deficient in both ABCA1 and ACAT2. J Lipid Res 46:2423-2431.

17. Can, T. P., Andresen, C. J., and Rudel, L. L. 1993. Enzymatic determination of triglyceride, free cholesterol, and total cholesterol in tissue lipid extracts. Clin Biochem 26:39-42.

18. Brunt, E. M., Janney, C. G., Di Bisceglie, A. M., Neuschwander-Tetri, B. A., and Bacon, B. R. 1999. Nonalcoholic steatohepatitis: a proposal for grading and staging the histological lesions. Am J Gastroenterol 94:2467-2474.

19. Kleiner, D. E., Brunt, E. M., Van Natta, M., Behling, C., Contos, M. J., Cummings, O. W., Ferrell, L. D., Liu, Y. C., Torbenson, M. S., Unalp-Arida, A., et al. 2005. Design and validation of a histological scoring system for nonalcoholic fatty liver disease. Hepatology 41:1313-1321.

20. Bell, T. A., 3rd, Brown, J. M., Graham, M. J., Lemonidis, K. M., Crooke, R. M., and Rudel, L. L. 2006. Liver-specific inhibition of acyl-coenzyme a:cholesterol acyltransferase 2 with antisense oligonucleotides limits atherosclerosis development in apolipoprotein B100-only low-density lipoprotein receptor−/− mice. Arterioscler Thromb Vasc Biol 26:1814-1820.

21. Kavanagh, K., Fairbanks, L. A., Bailey, J. N., Jorgensen, M. J., Wilson, M. Zhang, L. Rudel, L. L., and Wagner, J. D. 2007. Characterization and heritability of obesity and associated risk factors in vervet monkeys. Obesity. 15:1666-1674.

22. Muniyappa, R., Lee, S., Chen, H., and Quon, M. J. 2008. Current approaches for assessing insulin sensitivity and resistance in vivo: advantages, limitations, and appropriate usage. Am J Physiol Endocrinol Metab 294:E15-26.

23. Parini, P., Davis, M., Lada, A. T., Erickson, S. K., Wright, T. L., Gustaffson, U., Sahlin, S., Einarsson, C., Ericksson, M., Angelin, B., Tomoda, H., Omura, S., Willingham, M. C., and Rudel, L. L. 2004. ACAT2 is localized to hepatocytes and is the major cholesterol-esterifying enzyme in human liver. Circulation. 110: 2017-2023.

24. Rudel, L. L., Davis, M., Sawyer, J., Shah, R., and Wallace, J. 2002. Primates highly responsive to dietary cholesterol up-regulate hepatic ACAT2, and less responsive primates do not. J Biol Chem 277:31401-31406.

24. Lee, R. G., Willingham, M. C., Davis, M. A., Skinner, K. A., and Rudel, L. L. 2000. Differential expression of ACAT1 and ACAT2 among cells within liver, intestine, kidney, and adrenal of nonhuman primates. J Lipid Res 41:1991-2001.

25. Ioannou, G. N., Morrow, 0.B., Connole, M. L., and Lee, S. P. 2009. Association between dietary nutrient composition and the incidence of cirrhosis or liver cancer in the United States population. Hepatology 50:175-184.

26. Puri, P., Baillie, R. A., Wiest, M. M., Mirshahi, F., Choudhury, J., Cheung, O., Sargeant, C., Contos, M. J., and Sanyal, A. J. 2007. A lipidomic analysis of nonalcoholic fatty liver disease. Hepatology 46:1081-1090.

27. Fujita, K., Nozaki, Y., Wada, K., Yoneda, M., Fujimoto, Y., Fujitake, M., Endo, H., Takahashi, H., Inamori, M., Kobayashi, N., et al. 2009. Dysfunctional very-low-density lipoprotein synthesis and release is a key factor in nonalcoholic steatohepatitis pathogenesis. Hepatology 50:772-780.

28. Haemmerle, G., Zimmermann, R., and Zechner, R. 2003. Letting lipids go: hormone-sensitive lipase. Current Opinion in Lipidology 14:289-297.

29. Fabbrini, E., Sullivan, S., and Klein, S. Obesity and nonalcoholic fatty liver disease: Biochemical, metabolic, and clinical implications. Hepatology 51:679-689.

30. Willner, I. R., Waters, B., Patil, S. R., Reuben, A., Morelli, J., and Riely, C. A. 2001. Ninety patients with nonalcoholic steatohepatitis: insulin resistance, familial tendency, and severity of disease. Am J Gastroenterol 96:2957-2961.

31. Fabbrini, E., Mohammed, B. S., Magkos, F., Korenblat, K. M., Patterson, B. W., and Klein, S. 2008. Alterations in adipose tissue and hepatic lipid kinetics in obese men and women with nonalcoholic fatty liver disease. Gastroenterology 134:424-431.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for the treatment and or management of steatosis in a patient in need thereof, comprising administration of an effective amount of a steroyl-O-acyltransferase inhibitor, said inhibitor being effective to reduce cholesterol ester accumulation, thereby inhibiting or reducing symptoms associated with steatosis.

2. A method for treatment of a lysosomal acid lipase deficiency disorder in a patient in need thereof, the method comprising administering to said patient a selective steroyl-O-acyltransferase 2 (SOAT2) inhibitor in an amount effective to inhibit cholesterol ester accumulation in said patient.

3. The method of claim 2, wherein said lysosomal acid lipase disorder is selected from the group consisting of NAFLD, NASH, alcoholic liver disease, cryptogenic cirrhosis, Niemann-Pick disease Type C, Chanarin Dorfman syndrome, Abetalipoproteinemia, Familial hypobetalipoproteinemia, Citrullinemia type II, Familial partial lipodystrophy type 2, Familial partial lipodystrophy type 3, Congenital generalized lipodystrophy, Neutral lipid storage disorder, Cholesterol ester storage disease, Medium-chain acylcoenzyme-A dehydrogenase deficiency, Very long-chain acyl-CoA dehydrogenase deficiency, and Long-chain 3-hydroxyacyl-CoA dehydrogenase deficiency, cholesterol ester storage disease and Wolman disease.

4. The method of claim 3, wherein said disorder is cholesterol ester storage disease or Wolman disease.

5. The method of claim 1, wherein said inhibitor is an inhibitory nucleic acid molecule.

6. The method of claim 5, wherein said inhibitor is selected from the group consisting an antisense oligonucleotide inhibitor, an siRNA and a miRNA.

7. The method of claim 1, wherein said selective SOAT2 inhibitor is pyripyropene A, a pyripyropene derivative or a pharmaceutically acceptable salt, solvate or hydrate thereof.

8. The method of claim 7, wherein said pyripyropene derivative is selected from the group consisting of PRD 001, PRD 002, PRD 003, PRD 004, PRD 005, PRD 006, PRD 007, PRD 008, PRD 009, PRD 010, PRD 011, PRD 012, PRD 013, PRD 014, PRD 015, PRD 016, PRD 017, PRD 018, PRD 019, PRD 020, PRD 021, PRD 022, PRD 023, PRD 024, PRD 025, PRD 026, PRD 027, PRD 028, PRD 029, PRD 030, PRD 031, PRD 032, PRD 034, PRD 035, PRD 036, PRD 037, PRD 038, PRD 039, PRD 040, PRD 041, PRD 042, PRD 043, PRD 044, PRD 045, PRD 046, PRD 047, PRD 048, PRD 049, PRD 050, PRD 051, PRD 052, PRD 053, PRD 054, PRD 055, PRD 056, PRD 057, PRD 058, PRD 059, PRD 060, PRD 061, PRD 062, PRD 063, PRD 064, PRD 065, PRD 066, PRD69, PRD 70, PDR 71, PRD 73, PRD 74, PRD 79, PRD 80, PRD 81, PRD 075, PRD 084, PRD 085, PRD 087, PRD 090, PRD 092, PRD 093, PRD 095, PRD 096, PRD 098, PRD 100, PRD 102, PRD 103, PRD 104, PRD 105, PRD 106, PRD 107, PRD 108, PRD 109, PRD 110, PRD 111, PRD 112, PRD 119, PRD121, PRD122, PRD123, PRD125, PRD126, PRD143, PRD155, PRD156, PRD157, PRD158, PRD159, PRD160, PRD161, PRD162, PRD163, PRD164, PRD166, PRD167, PRD177, PRD180, PRD181, PRD186, and PRD187.

9. The method of claim 8, wherein said pyripyropene derivative is 1,11-O-o-methylben zylidene-7-O-p-cyanobenzoyl-1,7,11-trideacetylpyripyropene A, PRD125.

10. The method of claim 2, further comprising assessing the inhibitory effect resulting from administration of said inhibitor on cholesterol accumulation in said patient liver and/or serum.

11. The method of claim 10, wherein assessing the inhibitory effect of said SOAT2 inhibitor comprises determining cholesterol levels in liver and/or serum.
